(19) United States
(12) Patent Application Publication
(10) Pub. No.: US 2011/0153022 A1
(43) Pub. Date: Jun. 23, 2011

(54) BALLOON-ASSISTED ANNULUS REPAIR

(75) Inventors: Wamis Singhatat, Malvern, PA (US); Nigel G. Smith, Norwich (GB); Garland W. Fussell, Thorndale, PA (US); Michael F. Keane, Downingtown, PA (US); Anthony M. Lowman, Wallingford, PA (US)

(73) Assignees: SYNTHES USA, LLC, West Chester, PA (US); DREXEL UNIVERSITY, Philadelphia, PA (US)

(21) Appl. No.: 12/994,340

(22) PCT Filed: May 29, 2009
(86) PCT No.: PCT/US09/45690

§ 371 Date: Feb. 7, 2011

Related U.S. Application Data
(60) Provisional application No. 61/057,627, filed on May 30, 2008.

(51) Int. Cl. A61F 2/44

(52) U.S. Cl. 623/17.16

(57) ABSTRACT

The present invention is directed to an inflatable annulus repair device (10) for sealing an annulus defect located in an annulus fibrosis of an intervertebral disc space. In use, the inflatable device is introduced, in a first non-expanded state, preferably via a cannula (100) into the annulus defect. After the inflatable device has been properly positioned, a filler material (11) is injected into the inflatable device to expand the device to a second expanded state. In the second expanded state, the inflatable annulus repair device seals the annulus defect, secures its position within the annulus defect to thereby limit or prevent migration, and applies a compression force to a captured portion of the annulus adjacent to the defect. The inflatable device is preferably filled with a liquid that solidifies into an elastic material within the device.

### CROSS-REFERENCE TO RELATED APPLICATIONS

This application claims the benefit of U.S. Provisional Patent Application No. 61/057,627, filed on May 30, 2008, entitled “BALLOON-ASSISTED ANNULUS REPAIR,” the contents of which is incorporated in its entirety by reference herein.

### BACKGROUND OF THE INVENTION

Back pain is suffered by millions of Americans. A common type of back pain is caused by ruptured or herniated discs of the spine. Rupture or herniation of a disc results in the outer wall of an intervertebral disc (i.e., the annulus fibrosis) becoming weakened. As a result, the annulus fibrosis of the disc tears, allowing the soft inner part of the disc (i.e., the nucleus pulpous) to push out of the annulus. Once the nucleus pulpous extends past the regular margin of the annulus fibrosis, the nucleus pulpous can press against sensitive nerve tissues in the spinal structure (or anatomy), potentially resulting in back and leg pain. One treatment for relieving back pain is a discectomy, wherein parts of the damaged disc are removed to relieve pressure on the nerve tissue and alleviate pain. The surgery generally involves a small incision in the skin over the spine, removal of some ligament and bone material to access the disc and removal of some of the disc material. One problem generally associated with a discectomy, is that nerve root impingement is treated by removing a portion of the herniated disc while leaving the remaining disc in a weakened state with the possible risk of reherniation. In addition, discectomy may lead to a decrease in disc height, which can lead to further degeneration of the treated disc.

Thus, it is desirable to construct an annulus repair device that repairs the patient's annulus following a discectomy or other procedure in a minimally invasive manner.

### BRIEF SUMMARY OF THE INVENTION

A preferred embodiment of the present invention is directed to an inflatable annulus repair device for repairing and/or sealing an annulus defect located in an annulus fibrosis of an intervertebral disc space. In use, the inflatable device is introduced, in an unexpanded state, preferably via a cannula, into the annulus defect. After the inflatable device has been positioned, a filler material is injected into the inflatable device to expand the device to a second, expanded state. In the second, expanded state, the inflatable annulus repair device seals the annulus defect and secures its position within the annulus defect to thereby limit or prevent migration. The inflatable device is preferably filled with a liquid that solidifies into an elastic solid within the device.

The inflatable annulus repair device preferably includes a central body portion adapted to be positioned in the annulus defect, an intradiscal region adapted to be positioned within the intervertebral disc space, and an extradiscal region adapted to be positioned exterior the annulus portion. In use, the inflatable annulus repair device is inflatable from a first non-expanded state to a second expanded state. In the second expanded state the central body, intradiscal region and extradiscal region are all inflated to a larger, radial diameter to seal the annulus defect and to limit or prevent migration of the implant with respect to the annulus defect.

The inflatable annulus repair device may further include one or more deployable secondary fixation members or laterally expandable elements to enhance securement of the inflatable annulus repair device to the annulus wall to limit or prevent migration of the device. The deployable secondary fixation members or laterally expandable elements may be located adjacent to the intradiscal region to contact an inner surface of the annulus fibrosis upon inflation. Alternatively and/or in addition, the deployable secondary fixation members or laterally expandable elements may be located adjacent to the extradiscal region to contact the outer surface of the annulus fibrosis upon inflation. The deployable secondary fixation members or laterally expandable elements may be in the form of one or more arms. The deployable secondary fixation members or laterally expandable elements may be circumferentially disposed about the inflatable annulus repair device.

The inflatable annulus repair device may further include one or more retaining members formed on an outer surface of the central body to enhance fixation of the implant to the annulus wall. The retaining members may be in the form a projection, a ridge, etc. Alternatively, the retaining members may be a pore or hole so that, upon injection, the filler material can seep out of the inflatable annulus repair device to interact and/or interlock with the surrounding tissue.

The inflatable annulus repair device preferably is filled with a fluid that solidifies into an elastic solid at body temperature. More preferably, the inflatable annulus repair device may be filled with a thermogelling or phase transforming polymer.

The inflatable annulus repair device is preferably configured so that during inflation, the intradiscal and/or extradiscal regions expand toward the central body to further contact the interior and/or exterior annular walls of the annulus fibrosis to apply a compression force to a captured portion of the annulus adjacent the defect in an implanted position.

The present invention in one preferred embodiment is further directed to a method for repairing an annulus defect located in an annulus fibrosis of an intervertebral disc space. The method includes using an inflatable annulus repair device having a central body, an intradiscal region and an extradiscal region. The method may include the steps of forming an incision, inserting a cannula into the incision so that a distal end of the cannula is positioned adjacent to the annulus defect, inserting the inflatable annulus repair device through the cannula and at least partially into the annulus defect so that the intradiscal region extends through the annulus defect and into the intervertebral disc space, and injecting a filler material into the inflatable annulus repair device to seal the annulus defect.

### BRIEF DESCRIPTION OF THE SEVERAL VIEWS OF THE DRAWINGS

The foregoing summary, as well as the following detailed description of the preferred embodiments of the application, will be better understood when read in conjunction with the appended drawings. For the purposes of illustrating the device of the present application, there is shown in the drawings preferred embodiments. It should be understood, however, that the application is not limited to the precise arrangements, configurations, features and instrumentalities shown. In the drawings:

FIG. 1A illustrates a top plan view of an inflatable annulus repair device in accordance with one aspect of the present invention, the inflatable annulus repair device being inserted in a first non-expanded state into an annulus defect located within an annulus fibrosis of an intervertebral disc space;

FIG. 1B illustrates a top plan view of the inflatable annulus repair device shown in FIG. 1A, the inflatable annulus repair device in a second expanded state within the annulus defect;

FIG. 2A illustrates a side elevational view of a second preferred embodiment of an inflatable annulus repair device, the inflatable annulus repair device being inserted in the first non-expanded state into an annulus defect located within an annulus fibrosis of an intervertebral disc space;

FIG. 2B illustrates a side elevational view of the inflatable annulus repair device shown in FIG. 2A, the inflatable annulus repair device being inflated within the annulus defect;

FIG. 2C illustrates a side elevational view of the inflatable annulus repair device shown in FIG. 2A, the inflatable annulus repair device illustrated in the second expanded state within the annulus defect and a cannula being removed from the annulus repair device;

FIG. 3 illustrates a top plan view of a third preferred embodiment of an inflatable annulus repair device in a second expanded state, with a portion of a central body portion extending out of the annulus defect shown in dashed line-type;

FIG. 4 illustrates a top, plan view of a fourth preferred embodiment of an inflatable annulus repair device in a second expanded state;

FIG. 5 illustrates a top plan view of a fifth preferred embodiment of an inflatable annulus repair device in a second expanded state;

FIG. 6 illustrates a side elevational view of a sixth preferred embodiment of an inflatable annulus repair device in a second expanded state;

FIG. 7 illustrates a side elevational view of a seventh preferred embodiment of an inflatable annulus repair device in a second expanded state;

FIG. 8 illustrates a side elevational view of an eighth preferred embodiment of an inflatable annulus repair device in a second expanded state;

FIG. 9 illustrates a side elevational view of a ninth preferred embodiment of an inflatable annulus repair device in a second expanded state;

FIG. 10 illustrates a side elevational view of a tenth preferred embodiment of an inflatable annulus repair device in a second expanded state;

FIG. 11 illustrates a side elevational view of an eleventh preferred embodiment of an inflatable annulus repair device in a second expanded state;

FIG. 12 illustrates a side elevational view of a twelfth preferred embodiment of an inflatable annulus repair device in a second expanded state;

FIG. 13 illustrates a side elevational view of a thirteenth preferred embodiment of an inflatable annulus repair device in a second expanded state; and

FIG. 14 illustrates a side elevational view of a fourteenth preferred embodiment of an inflatable annulus repair device in a second expanded state.

### DETAILED DESCRIPTION OF THE INVENTION

Certain terminology is used in the following description for convenience only and is not limiting. The words “right”, “left”, “lower” and “upper” designate directions in the drawings to which reference is made. The words “inwardly” and “outwardly” refer to directions toward and away from, respectively, the geometric center of the annulus repair device and designated parts thereof. The words, “anterior”, “posterior”, “superior”, “inferior” and related words and/or phrases designate preferred positions and orientations in the human body to which reference is made and are not meant to be limiting. The terminology includes the above-listed words, derivatives thereof and words of similar import.

Certain exemplary embodiments of the invention will now be described with reference to the drawings. In general, the present invention is directed to an annulus repair device 10, 10′, 10″, 10′″, 10″″, 10′″″, 10″″″, 10′″″″, 10″″″″, 10′″″″″, 10″″″″″, 10′″″″″″, 10″″″″″″ (collectively 10X) and to a surgical method or procedure for inserting the same within an opening or tear (collectively referred to herein as an annulus defect D) formed in the annulus fibrosis F of an intervertebral disc. More specifically, preferred embodiments of the present invention are directed to an inflatable annulus repair device 10X and associated surgical method or procedure for inserting the inflatable annulus repair device 10X within an annulus defect D so that the annulus defect D can be sealed. That is, the preferred embodiments of the present invention are directed to the inflatable annulus repair device 10X and surgical method or procedure for repairing an annulus defect D in the annulus fibrosis F of the intervertebral disc space S, post-discectomy or other related procedure, by deploying, within the annulus defect D, the inflatable annulus repair device 10X to fill, seal, secure, and/or repair the defect D. The inflatable annulus repair device 10X is preferably filled with a fluid (e.g., polymer) 11 that transforms into an elastic solid within the device 10X so that together, the inflatable annulus repair device 10X and the elastic core, repair the defect D and generally seal the disc S from further reherniation or deflation. In addition, the arrangement of injectable filler material 11 and inflatable annulus repair device 10X preferably hydraulically pressurizes or fills against a back pressure created by the remaining nucleus material N located within the intervertebral disc space S.

In use, the inflatable annulus repair device 10X is inserted into the annulus defect D in a first non-expanded state. The device 10X is then preferably inflated and/or expanded to a second expanded state to seal the annulus defect D to limit or prevent any additional material from the intervertebral disc space S from leaking out of the annulus defect D. The device 10X is also preferably sized and configured, in the second expanded state, to limit or prevent migration of the device 10X with respect to the annulus defect D. That is, in use, a surgeon preferably forms an incision and inserts a cannula 100 into the incision so that a distal end of the cannula 100 is positioned adjacent to the annulus defect D so that the surgeon can visualize and/or access the annulus defect D. Thereafter, the surgeon preferably inserts the inflatable annulus repair device 10X at least partially into the annulus defect D so that a central body portion 12 extends through the annulus defect D and into the intervertebral disc space S. Next, the surgeon preferably injects a filler material 11, preferably via a catheter 110, into the inflatable annulus repair device 10X, which results in the device 10X inflating and/or expanding to the second expanded state, which in turn preferably seals the annulus defect D and limits or prevents migration of the device 10 with respect to the defect D. The filler material 11 may be a thermogelling or phase transforming polymer, as will be described in greater detail below, that solidifies to an elastic solid after being injected into the annulus repair device 10X. Alternatively, the filler material 11 may be any other material known in the art, as will be described in greater detail below. If necessary, the device 10X may be sealed to prevent leaking of the filler material 11 from the device 10X. Thereafter, the cannula 100 and catheter 110 are removed and the incision is closed. The cannula 100 and inflatable annulus repair device 10X may be inserted via a posterior approach, an anterior approach, a lateral approach, an anterior-lateral approach, a posterior-lateral approach, by nearly any approach that permits a surgeon to gain access to the defect D in the annulus F, as will be apparent to one having ordinary skill in the art.

As will be described in greater detail below, while the inflatable annulus repair device 10X and preferred surgical method or procedure of the present invention is described in connection with and generally may be used for sealing the annulus defect D in the intervertebral disc space S, it will be generally understood by one of ordinary skill in the art, that the inflatable annulus repair device 10X and surgical method or procedure may be equally applicable in other surgical procedures in which a surgeon desires to seal a defect or repair damage to tissue including, but not limited to, for use in connection with a nucleus replacement, etc.

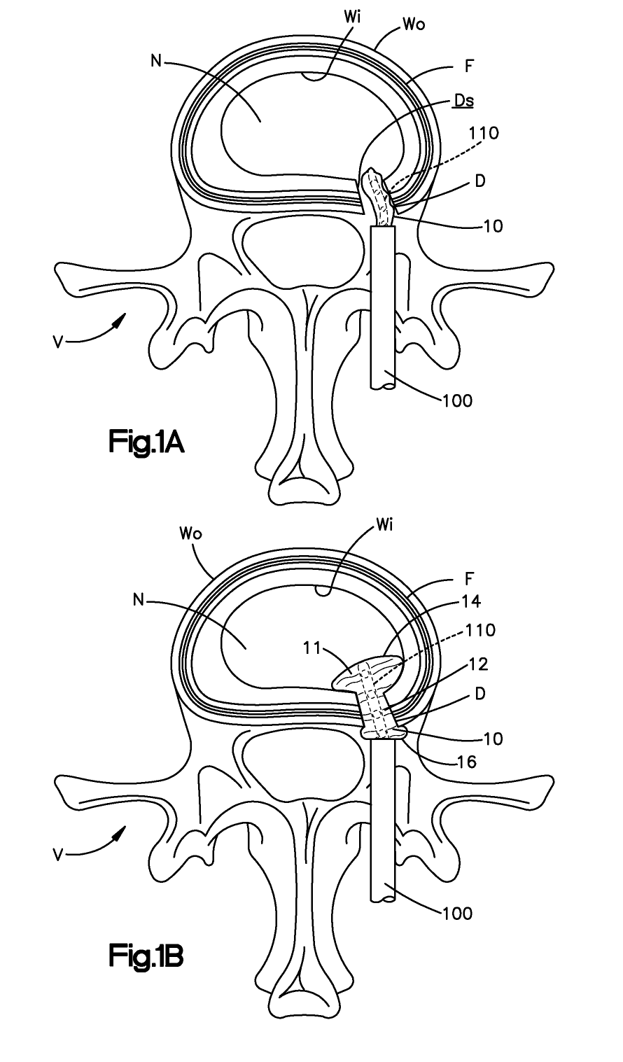

Referring to FIGS. 1A and 1B, in a first preferred embodiment, the inflatable annulus repair device 10 includes an inner cavity for receiving an injectable filler material 11. That is, in use, after a discectomy or other similar procedure has been performed, an inflatable annulus repair device 10 is introduced, in a first non-expanded state, preferably via a cannula 100 into an annulus defect D located in the annulus fibrosis F of an intervertebral disc space S. After the inflatable annulus repair device 10 has been introduced into the annulus defect D, the surgeon injects a filler material 11 into the inner cavity of the inflatable annulus repair device 10 thereby expanding the inflatable annulus repair device 10 to a second, expanded state. In the second, expanded state, the inflatable annulus repair device 10 seals the annulus defect D and preferably secures its position within the annulus defect D to thereby limit or prevent migration of the inflatable annulus repair device 10. The inflatable annulus repair device 10 is insertable into the annulus defect D in a first non-expanded state via a minimally invasive procedure, expandable to a second expanded state in which the inflatable annulus repair device 10 conforms to a variety of defect shapes and/or sizes, and is able to set in place to begin a natural interaction with the surrounding nucleus N and annulus F tissue.

As will be appreciated by one of ordinary skill in the art, the inflatable annulus repair device 10 and surgical method or procedure of the present invention is not limited for use in connection with a discectomy. Rather, the inflatable annulus repair device 10 and surgical method or procedure of the present invention may be used to surgically repair an annulus defect D regardless if a discectomy has been performed. The annulus repair device 10 may be utilized to repair the defect D formed as the result of a surgical procedure or may be utilized to repair a naturally occurring defect D in the annulus fibrosis F of the intervertebral disc space S.

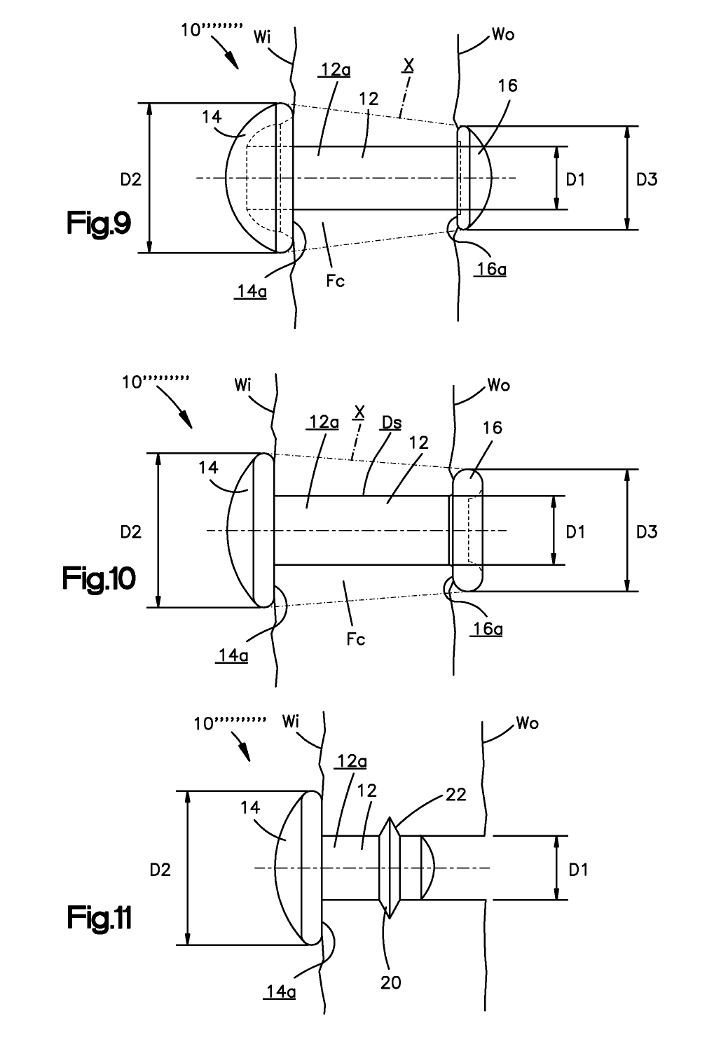

Referring to FIGS. 1A-14, upon expansion, the inflatable annulus repair device 10X preferably includes a central body 12 positioned within the annulus defect D and an intradiscal region 14 distal to and preferably larger in radial size than the central body 12. That is, the inflatable annulus repair device 10X preferably includes the intradiscal region 14 that is insertable into and through the annulus defect D and into the nucleus pulpous region N of the intervertebral disc space S in the first non-expanded state. Upon expansion, the intradiscal region 14 preferably expands to a larger diameter than the central body 12 and the annulus defect D to thereby assist, in combination with the central body 12, in sealing the annulus defect D and limiting or preventing migration of the inflatable annulus repair device 10X back through the annulus defect D. The expanded intradiscal region 14 may take on nearly any shape including, but not limited to, spherical, circular, rectangular, oval, mushroom-shaped, etc. when expanded.

Additionally, the inflatable annulus repair device 10X of the preferred embodiments generally includes an expanded extradiscal region 16 proximal to and preferably larger in radial size than the central body 12. That is, the inflatable annulus repair device 10X also includes the extradiscal region 16 that, upon expansion, is sized and configured to expand to a larger diameter than the central body 12 and the annulus defect D to thereby assist, in combination with the central body 12 and preferably the intradiscal region 14, with sealing of the annulus defect D and to limit or prevent migration of the inflatable annulus repair device 10X through the annulus defect D. The expanded extradiscal region 16 may take on any shape known in the art including, but not limited to, spherical, circular, rectangular, oval, mushroom-shaped, etc. when expanded. The expanded diameter of the extradiscal region 16 may be larger than, smaller than or equal to the expanded diameter of the intradiscal region 14. The annulus repair device 10X of the preferred embodiments is not limited to inclusion of the extradiscal region 16 and may include only the central body 12 and the intradiscal region 14 (See FIGS. 8, 11, 13 and 14).

Upon expansion, the inflatable annulus repair device 10X preferably assumes and maintains a generally defined shape that creates a seal and/or interlock with an inner annulus wall W, of the annulus fibrosis F that is mechanically stable under physiologic loading of the intervertebral disc. The inflatable annulus repair device 10X is preferably injected with a fluid, such as, for example, a thermogelling or phase transforming polymer, as will be described in greater detail below. As will be readily appreciated by one of ordinary skill in the art, the amount of expansion can be varied by adjusting the inflation volume and/or pressure.

During delivery and subsequent expansion of the inflatable annulus repair device 10X, the device 10X may be brought into close contact with the annular walls Wᵢ, Wₒ and/or the annular defect D, and the device 10X may maintain close contact with the annulus walls Wᵢ, Wₒ and/or the annular defect D under physiologic loading of the disc, thereby creating a seal across the annular defect D between the intradiscal and extradiscal regions.

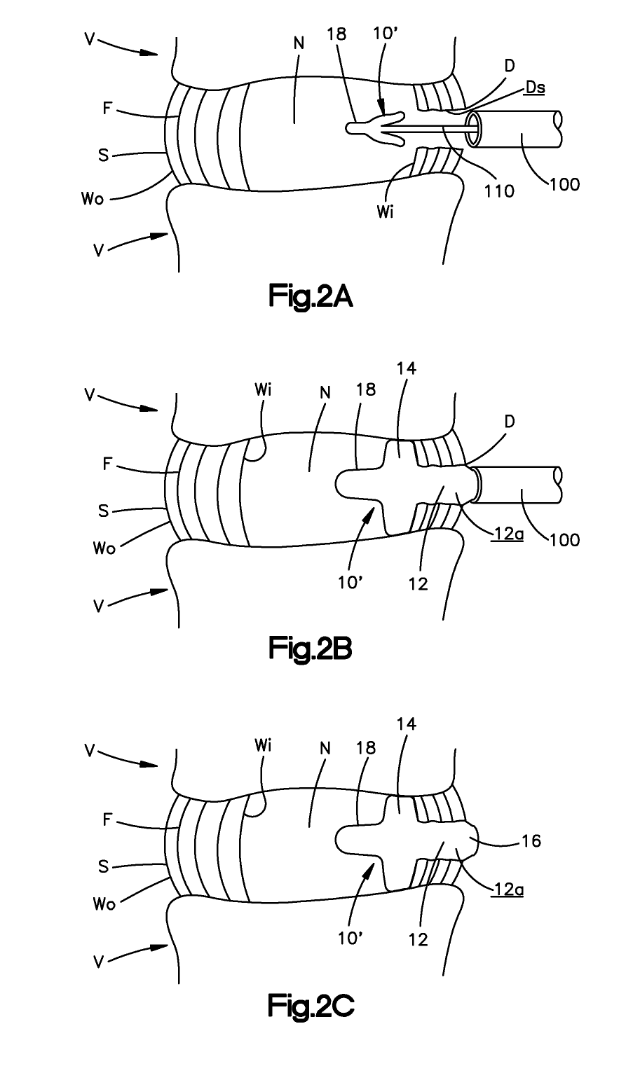

Referring to FIGS. 1A, 1B and 2A, a catheter 110 is preferably provided and/or is insertable into the inflatable annulus repair device 10X so that the filler material 11 may be injected into the device 10 to inflate and/or expand the device 10X from the first non-expanded state to the second expanded state. The catheter 110 is preferably sized and configured to extend from the intradiscal region 14 to the extradiscal region 16 of the device 10 so that the intradiscal and extradiscal regions 14, 16 can be simultaneously inflated and/or expanded. A method is preferably utilized to manually apply axial tension to the catheter-inflatable device construct during and/or after deployment of the intradiscal region 14 of the inflatable annulus repair device 10X. The axial tension is achieved by radial expansion of the device 10X and is dependent on sizing and pressure.

The geometry of the inflatable annulus repair device 10X, upon expansion, preferably imparts compression to the interior and/or exterior annular walls Wᵢ, Wₒ along the axis of the inflatable annulus repair device 10 between the intradiscal and extradiscal regions 14, 16, as well as imparts preferably radial compression through the central body portion 12 of the inflatable device 10X throughout the length of the annular defect D between the interior and the exterior of the annulus defect D. Radially compression may be provided by determining the size (e.g., length) of the defect D and using a device 10X that has a longitudinal length, post inflation, slightly less than the longitudinal size of the defect D so that a compressive force is applied to the inner and outer annular walls Wᵢ, Wₒ.

The inflatable annulus repair device 10X may include variable properties to suit a variety of applications. The inflatable annulus repair device 10X is preferably designed to accommodate a variety of annular wall geometries. For example, the inflatable annulus repair device 10X may be manufactured from a highly compliant material so that the inflatable annulus repair device 10X of the preferred embodiments is able to conform to a variety of anatomical shapes, sizes and thicknesses to optimize sealing and closure of the annulus defect D. Alternatively, the inflatable annulus repair device 10X may be manufactured from a low-compliant or non compliant material for achieving and maintaining a predetermined shape upon expansion. Examples of highly compliant material for manufacturing the inflatable annulus repair device 10X include polycarbonate urethanes such as, for example, Bionates, Carbosil, etc.; polyether urethane silicone; polyester urethanes such as, for example, Estanes, etc.; silicone elastomers; latex natural rubber; nirtrile latex rubbers; etc. Examples of less compliant material for manufacturing the inflatable annulus repair device 10X include polycarbonate urethanes such as, for example, Bionate 75D Grade, PEBAX, etc.; nylon; low density polyolefins such as, for example, polyethylene, polypropylene, etc. Examples of non compliant material for manufacturing the inflatable annulus repair device 10X include polyetheretherketone (PEEK), polyetherketoneketone (PEKK); polyethylene terephthalate (PET); etc.

Moreover, referring to FIG. 1, the distal intradiscal region 14 of the inflatable annulus repair device 10 may be manufactured from a material with low-compliance while the proximal extradiscal region 16 may be manufactured from a material with high-compliance, such that the expansion of the inflatable annulus repair device 10 initially expands the intradiscal region 14 and subsequently expands the extradiscal region 16, whereby further expansion of the extradiscal region 16 may draw the intradiscal region 14 into closer contact with and/or compression against the inner annular wall Wᵢ. Alternatively, referring to FIG. 7, the inflatable annulus repair device 10′″″ may be manufactured with an intradiscal region 14 having high compliance while the extradiscal region 16 may be constructed from a material with low compliance. Such a configuration can assist the positioning of the inflatable annulus repair device 10′″″ in the defect D. If desired, the high compliance intradiscal region 14 can be expanded prior to the low compliance extradiscal region 16 by applying an external force to the extradiscal region 16 such as, for example, via the cannula 100. In this manner, the intradiscal region 14 inflates first, then by reducing the force applied to the extradiscal region 16 via the cannula 100, the extradiscal region 16 would expand upon injection of additional filler material.

Referring to FIGS. 2A-2C, the inflatable annulus repair device 10′ of a second preferred embodiment may additionally include an inflatable projection 18 extending from and/or formed on the intradiscal region 14 to fill some or all of an intradiscal cavity, such as a cavity resulting from the removal of some or all of the nucleus N during a discectomy procedure. That is, the intradiscal region 14 may include the projection 18 sized and configured, upon expansion, to fill a space or cavity left in the intervertebral disc space S as a result of, for example, the discectomy. The amount of injected filler material 11 can be varied by adjusting the inflation volume and/or pressure. The projection 18 is not limited to having any specific size and/or shape and is preferably relatively compliant to fill potentially irregular shaped cavities in the nucleus resulting from removal of material.

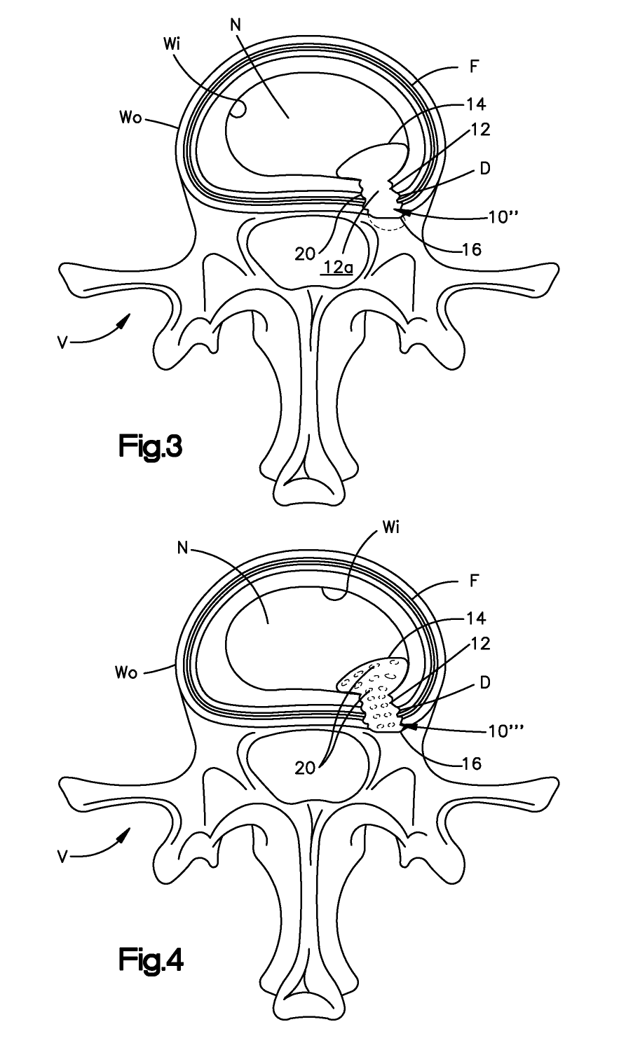

In addition, referring to FIGS. 3, 4, 8 and 11, in the third, fourth, eighth and eleventh preferred embodiments, the inflatable annulus repair device 10″, 10′″, 10′″″″, 10″″″″″ includes one or more retaining members 20 to provide additional fixation to the annulus fibrosis F. For example, as best shown in FIG. 3, the retaining members 20 may be in the form of one or more projections formed on the outer surface of the central body 12, the intradiscal region 14 or the extradiscal region 16 to enhance securement of the inflatable annulus repair device 10″ to the annulus wall and prevent or limit migration. As best shown in FIGS. 8 and 11, the retaining members 20 may be in the form of one or more ridges 22 formed on the outer surface of the central body 12 to enhance securement of the inflatable annulus repair device 10′″″″, 10″″″″″ to the annulus wall to limit or prevent migration. As best shown in FIG. 4, the inflatable annulus repair device 10′″ may be configured as a “weeping” balloon. That is, the inflatable annulus repair device 10′″ may include a plurality of pores or holes so that, upon injection, the filler material 11 can seep out of the inflatable annulus repair device 10′″ and interact with surrounding tissue. The inflatable annulus repair device 10′″ is particularly useful in combination with a tissue adhesive filler material 11 to enable the inflatable annulus repair device 10′″ to fully integrate with the surrounding annulus tissue.

The inflatable annulus repair device 10X may further incorporate an adhesive polymer on, for example, an external surface 12a of the central body portion 12, a first disc surface 14a of the intradiscal region 14 and/or a second disc surface 16a of the extradiscal region 16, so that the device 10X adheres to at least portions of the side surfaces Dₛ of the defect D, the inner annular wall W, and/or the exterior annular wall Wₒ, respectively.

Furthermore, referring to FIGS. 3, 4, 8 and 11, a cutting device (not shown) may be provided for cutting off any excess length of the inflatable annulus repair device 10X that protrudes externally to the defect D so that the inflatable annulus repair device 10X is generally flush with or recessed relative to the outer annular wall Wₒ after expansion and final positioning of the inflatable annulus repair device 10X. That is, as schematically shown in FIG. 3, a portion (shown in dashed line-type) of the central body portion 12 may extend out of the annulus defect D. After the filler material 11 is injected and hardens, the user may cut off the portion of the central body 12 that extends out of the annulus defect D so that the proximal end of the inflatable annulus repair device 10X is generally flush with or recessed relative to the outer annular wall Wₒ.

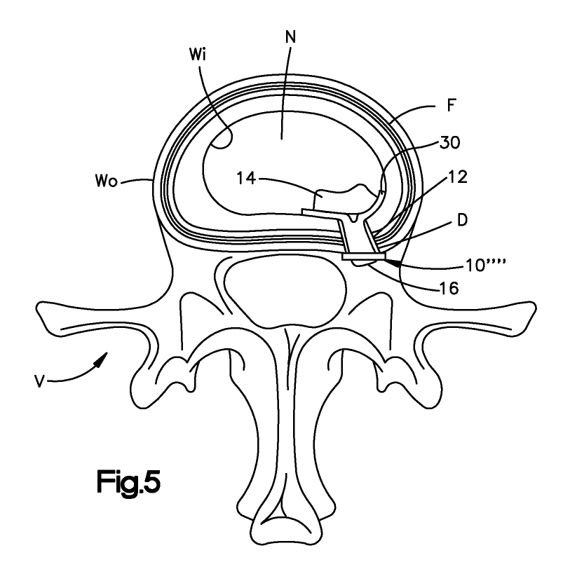

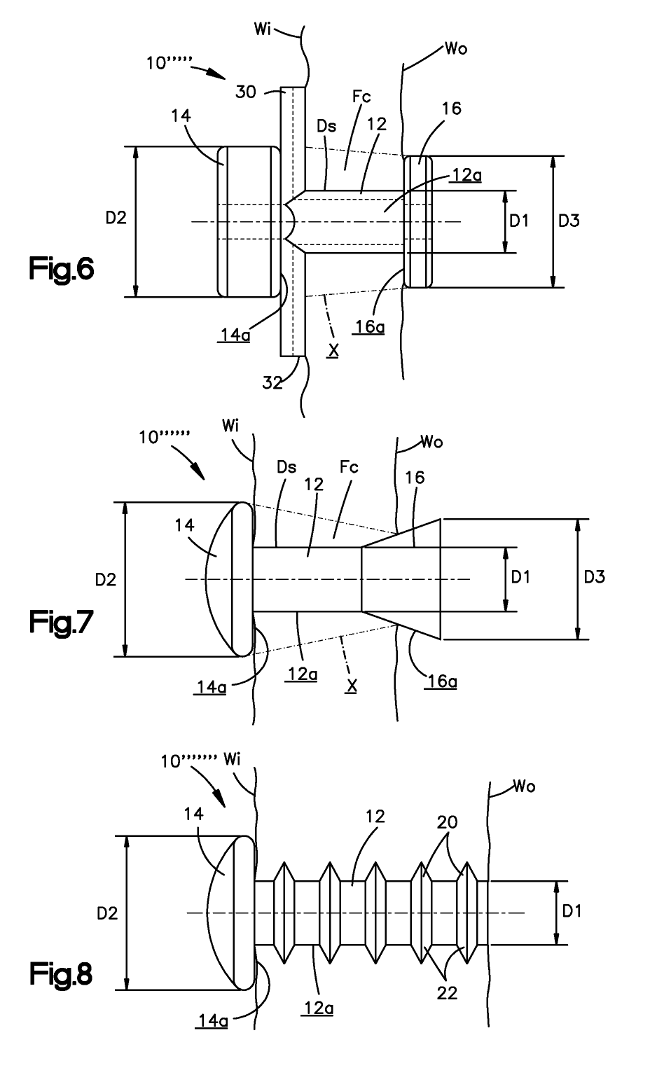

Alternatively and/or in addition, referring to FIGS. 5 and 6, the inflatable annulus repair device 10″″, 10′″″ of the fifth and sixth preferred embodiments may include a deployable secondary fixation member 30 to enhance securement of the inflatable annulus repair device 10″″, 10′″″ to the annulus walls Wᵢ, Wₒ to prevent or limit migration of the device 10″″, 10′″″. For example, as best shown in FIG. 6, the deployable secondary fixation member 30 of the annular repair device 10′″″ of the sixth preferred embodiment is comprised of one or more deployable arms 32 disposed adjacent to the intradiscal region 14 to provide additional fixation to the inner annular wall Wᵢ and/or to abut the annulus defect D from the interior of the intervertebral disc S. The deployable arm 32 is preferably inflated and/or expanded to a larger diameter than the central body 12 and the annulus defect D. More preferably, the deployable arm 32 is also inflated and/or expanded to a larger diameter than the intradiscal region 14. The deployable arms 32 may be circumferentially disposed about the inflatable annulus repair device 10′″″. Alternatively, the deployable secondary fixation member 30 may take on any other configuration to provide additional fixation to the inner or outer annular wall Wᵢ, Wₒ, to the defect D between the inner and outer annular walls Wᵢ, Wₒ and/or to abut the annulus defect D from the interior of the intervertebral disc S including, for example, a deployable sheath, etc. The secondary fixation member 30 may be configured to inflate simultaneously with, prior to or after inflation of the inflatable annulus repair device 10″″, 10′″″ of the fifth and sixth preferred embodiments.

The deployable secondary fixation member 30 can be provided instead of or in addition to the expanded intradiscal region 14. The inflatable annulus repair device 10X may further include one or more deployable secondary fixation members adjacent to the extradiscal region 16 of the inflatable annulus repair device 10X such that the extradiscal secondary fixation member (not shown) is positioned exterior to the outer annular wall Wₒ for additional fixation of the inflatable annulus repair device 10X to the annulus fibrosis F. The secondary fixation member 30 are preferably sized and configured to enable a compressive force to be imparted on the annulus fibrosis F upon actuation or deployment of the secondary fixation member 30.

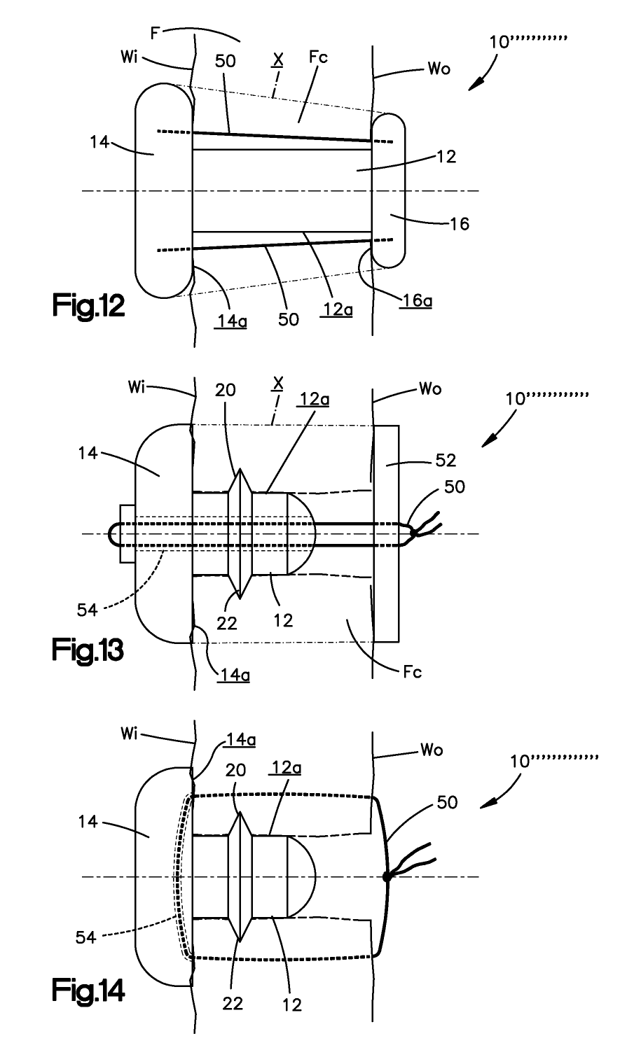

Referring to FIG. 12, the inflatable annulus repair device 10′″″″″″ of the twelfth preferred embodiment may include one or more bridging members such as, for example, sutures 50, that extend from the intradiscal region 14, through at least a portion of the annulus F, and to the extradiscal region 16 so that in use, a user can pull on or tension the suture 50 to bring the intradiscal region 14 into close contact with the inner annular wall Wᵢ and the extradiscal region 16 into close contact with the outer annular wall Wₒ and to apply an additional compressive force across the annular defect D between the intradiscal region 14 and the extradiscal region 16. The suture 50 is preferably tensioned after the filler material 11 has been injected and solidified within the inflatable annulus repair device 10′″″″″″.

Referring to FIG. 13, the inflatable annulus repair device 10″″″″″″ of the thirteenth preferred embodiment may include a shortened central body portion 12 that is attached via a bridging member such as, for example, a suture 50, to an extradiscal member 52, which can be brought into close contact with the outer annular wall Wₒ after the inflatable annulus repair device 10″″″″″″ is expanded, more preferably after the filler material 11 has been solidified within the inflatable annulus repair device 10″″″″″″. The suture 50 is preferably used to tension the inflatable annulus repair device 10″″″″″″ to impart a compressive force across the annular defect D between the intradiscal region 14 and the extradiscal member 52, at least in a captured portion F_{c} of the annulus F. The inflatable annulus repair device 10″″″″″″ may include a central bore 54 for receiving the suture 50. In use, the suture 50 is sized and configured to extend from the intradiscal region 14 of the inflatable annulus repair device 10″″″″″″ to the extradiscal member 52 so that the user can pull on or tension the suture 50 to apply a compressive force across the captured portion F_{c} adjacent the annular defect D between the intradiscal region 14 and the extradiscal member 52.

Alternatively, referring to FIG. 14, the inflatable annulus repair device 10′″″″″″″ of the fourteenth preferred embodiment may include a shortened central body portion 12 and a bridging member such as, for example, a suture 50. In this embodiment, the suture 50 preferably passes through the annulus F, through a bore 54 formed in the intradiscal region 14 of the inflatable annulus repair device 10′″″″″″″ and back through the annulus F so that the user can pull on or tension the suture 50 to bring the intradiscal region 14 into close contact with the inner annular wall Wᵢ and to apply a compressive force across the annular defect D.

Referring to the preferred embodiments of FIGS. 12, 13 and 14, the suture 50 can then be tied with any number of knots known in the field of surgery, including any of a variety of sliding knots, a surgeon's knot, and/or alternating half-hitches. The suture 50 may alternatively employ a pre-tied sliding knot such as disclosed in U.S. Provisional Patent Application No. 61/159,212, filed on Mar. 11, 2009, entitled “THREADABLE KNOT SOFT TISSUE DEFECT REPAIR DEVICE” the contents of which is incorporated in its entirety by reference herein.

The inflatable annulus repair device 10X may be of any size necessary to fill and seal the defect. For example, the device 10X may include a cross-sectional diameter of about 3 mm to about 10 mm, although other diameters are envisioned.

Referring to FIGS. 1A-14, the annulus repair device 10X of the preferred embodiments is mounted within the defect D in the annulus fibrosis F in an implanted position (FIGS. 1B and 2B-14) when the device 10X is in the expanded state. In the implanted position, an external surface 12a of the central body portion 12 contacts at least portions of side surfaces Dₛ of the defect D, a first disc surface 14a of the intradiscal region 14 contacts the inner annular wall Wᵢ of the annulus fibrosis F adjacent the defect D and a second disc surface 16a of the extradiscal region 16 may contact the outer annular wall Wₒ of the annulus fibrosis F adjacent the defect D. Further, in the implanted position, a captured portion F_{c} of the annulus fibrosis F, which is generally bounded by the first and second disc surfaces 14a, 16a, the external surface 12a and an imaginary surface X defined by connecting an edge of engagement between the first disc surface 14a and the outer annular wall Wₒ with an edge engagement between the second disc surface 16a and the inner annular wall Wᵢ. The captured portion Fc of the annulus fibrosis F is placed under compression to generally limit movement or migration of the device 10X from the implanted position. The retaining members 20 or certain embodiments further secure the device 10X within the defect D in the implanted position.

In the expanded state, the central body portion 12 has a first diameter D₁, the intradiscal region 14 has a second diameter D₂ and the extradiscal region 16 has a third diameter D₃. The second and third diameters D₂, D₃ are larger than the first diameter D₁ in the preferred embodiments of the device 10X. Arranging the diameters D₁, D₂, D₃ in this manner permits the device 10X to apply compression to the captured portion F_{c} in the implanted position. The second diameter D₂ is typically larger than the third diameter D₃ to limit the amount of the device 10X that protrudes from the outer annular wall Wo, but is not so limited and the second and third diameters D₂, D₃ may be substantially the same or the third diameter D₃ may be larger than the second diameter D₂. In addition, although the central body portion 12, intradiscal region 14 and extradiscal region 16 are indicated as having first, second and third diameters D1, D2, D3 in the preferred embodiments, this is not an indication that these portions of the devices 10X are limited to being generally cylindrically-shaped. For example, the central body portion 12, intradiscal region 14 and extradiscal region 16 may be generally rectangular-shaped, oval-shaped or have nearly any size and/or shape that enables insertion into the defect D in the first non-expanded state and engagement or mounting in and adjacent to the defect D in the expanded state and the implanted position.

The inflatable annulus repair device 10 may also include a valve for enabling injection of the filler material 11. Alternatively, the device 10X may include any other mechanism for sealing the device 10X to prevent the filler material 11 from leaking including, but not limited to, a suture, etc. Preferably, as will be described in greater detail below, the filler material 11 is a thermogelling or phase transforming polymer that solidifies within the device 10 in the expanded state.

The inflatable annulus repair device 10X maybe inflated with any filler material 11 known in the art including, but not limited to, saline, air, gas, water, etc. Preferably, however, the inflatable annulus repair device 10X is inflated with a thermogelling or phase transforming polymer. The utilization of a thermogelling or phase transforming polymer to expand the inflatable annulus repair device 10X enables the surgeon to optimize the inflatable annulus repair device 10X for his/her particular application by varying the properties of the device 10X and/or the cured thermogel or phase transforming polymer to possess structural properties similar to the natural annulus fibrosis F. In addition, utilization of a thermogelling or phase transforming polymer to expand the inflatable annulus repair device 10X enables the surgeon to implant the inflatable device 10X against a pressurized intradiscal environment due to the inflatable device's X and thermogelling or phase transforming polymer's ability to withstand inflation pressures substantially higher than that of the intradiscal space.

The thermogelling or phase transforming polymer for expanding the inflatable annulus repair device 10X may be comprised of a lower critical solution temperature (LCST) polymer that transitions at body temperature to an elastic solid to fill the inflatable device 10X, which is preferably compliant, so that the annulus defect D is sealed to prevent or limit reherniation or further depressurization of the intervertebral disc space S. U.S. patent application Ser. No. 10/837,082 to Lowman et al., filed on Nov. 4, 2004 and entitled “Thermogelling Polymer Blends for Biomaterial Applications”, which is hereby incorporated by reference in its entirety, discloses a thermogelling material in the form of a PniPaam copolymer that transitions slightly below body temperature into an elastic solid. By forming into an elastic solid within the inflatable annulus repair device 10X, disadvantages associated with leaking of a liquid filled container may be overcome.

Additional filler materials 11 that may be used in conjunction with the inflatable annulus repair device 10X of the preferred embodiments include, for example, ultraviolet (UV) curable materials and other cross linking chemistries. UV curing materials are typically acrylates or methacrylates. In use, UV curing materials can be injected into the inflatable device 10X to the desired fill pressure or volume at which point, a UV light source is used to initiate the curing reaction to form the final polymer material. In addition, different monomeric materials can be used to tailor the mechanical properties of the filler material 11. Because the reaction can be initiated at the surface and propagate inward, the risk of leaching of unreacted components is generally limited. The UV light source can be used at the surface of the annulus repair device 10X (i.e. at an injection port or hole formed in the inflatable annulus repair device 10X), inserted into the interior of the device 10X, or both. Additionally, a fiber optic component (not shown) may be incorporated in the device 10X to allow the UV light to be generated from within the device 10X, thus initiating the curing around the entire device 10X, and not just at the point of injection.

Other cross linking chemistries include the use of amine containing polymers and/or monomers that could be reacted by the addition of aldehyde containing materials. The aldehyde/amine reaction is generally used to crosslink materials for various applications. Additionally, due to the amine groups in the surrounding tissue, a porous device could be adhered to the surrounding tissue with this chemistry.

The UV curing process or the addition of an aldehyde or other cross linker can be used to seal the injection port of the inflatable annulus repair device 10X, regardless of whether the filler material 11 is a UV curable or cross linkable material. This can be used in lieu of or in conjunction with a mechanical closure system, such as suturing or clamping the port closed. With the ability to seal the port with a UV curing or cross linking system, the filler material 11 of the inflatable annulus repair device 10X may take a longer period of time to solidify and/or transform into its final state without concern of the material excreting from the opening.

Furthermore, for radiographic visualization, the inflatable annulus repair device 10X may possess a radiopaque character so that the surgeon can visualize the positioning and orientation of the device 10X as the device 10X is being inserted, both before and after filling. For example, the inflatable annulus repair device 10X may include printing with a radiopaque ink. Alternatively, the inflatable annulus repair device 10X may include a fiber or strand of radiopaque material. The filler material 11 may also incorporate radiopaque materials so that the entire device 10X can be visualized after implantation.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. An inflatable annulus repair device for repairing an annulus defect located in an annulus fibrosis surrounding an intervertebral disc space, the annulus defect having side surfaces extending between interior and exterior annular walls, the device comprising:
a central body portion having an external surface and a first diameter in an expanded state;
an intradiscal region having a first disc wall surface and a second diameter in the expanded state; and
an extradiscal region having a second disc wall surface and a third diameter in the expanded state, the central body portion, intradiscal region and extradiscal region insertable into the defect in a first non-expanded state, the external surface being in contact with the side surfaces, the first disc wall in contact with the interior annular wall and the second disc wall in contact with the exterior annular wall in an implanted position in the expanded state, the second and third diameters being larger than the first diameter in the expanded state, the first and second disc walls applying a compression force to a captured portion of the annulus adjacent the defect in the implanted position.

2. The inflatable annulus repair device of claim 1, wherein the central body, the intradiscal region and the extradiscal region are integrally formed as a balloon.

3. The inflatable annulus repair device of claim 1, wherein the intradiscal region includes one or more laterally expandable elements immediately adjacent to the central body, the expandable elements contacting the interior annular wall in the expanded state.

4. The inflatable annulus repair device of claim 3, wherein the extradiscal region includes one or more laterally expandable elements immediately adjacent to the central body, the expandable elements contacting the exterior annular wall in the expanded state.

5. The inflatable annulus repair device of claim 1, wherein the inflatable annulus repair device is filled with a fluid in the implanted position in the expanded state, the fluid solidifies into an elastic solid at body temperature.

6. The inflatable annulus repair device of claim 5, wherein the fluid is a thermogelling or phase transforming polymer.

7. The inflatable annulus repair device of claim 1, further comprising one or more retaining members formed on the external surface of the central body to enhance fixation of the implant to the side surfaces.

8. The inflatable annulus repair device of claim 7, wherein the one or more retaining members is selected from one of a projection or a ridge.

9. The inflatable annulus repair device of claim 1, further comprising a plurality of pores or holes so that, upon injection, a filler material seeps out of the device.

10. The inflatable annulus repair device of claim 1, further comprising one or more deployable secondary fixation members.

11. The inflatable annulus repair device of claim 10, wherein the one or more deployable secondary fixation members is located adjacent to the intradiscal region to contact the interior annular wall in the expanded state.

12. The inflatable annulus repair device of claim 10, wherein the one or more deployable secondary fixation members is located adjacent to the extradiscal region to contact the exterior annular wall in the expanded state.

13. The inflatable annulus repair device of claim 10, wherein the one or more deployable secondary fixation members is in the form of one or more arms.

14. The inflatable annulus repair device of claim 1, wherein the intradiscal region of the inflatable annulus repair device is manufactured from a highly compliant material and the extradiscal region of the inflatable annulus repair device is manufactured from a low compliant material.

15. The inflatable annulus repair device of claim 1, wherein the intradiscal region of the inflatable annulus repair device is manufactured from a low compliant material and the extradiscal region of the inflatable annulus repair device is manufactured from a high compliant material.

16. The inflatable annulus repair device of claim 1, further comprising an inflatable projection formed on the intradiscal region to fill at least a portion of an intradiscal cavity.

17. The inflatable annulus repair device of claim 1, further comprising one or more sutures extending from the intradiscal region to the extradiscal region.

18. The inflatable annulus repair device of claim 17, wherein the one or more sutures passes through the central body.

19. The inflatable annulus repair device of claim 17, wherein the one or more sutures passes through the annulus fibrosis.

20. A method for repairing an annulus defect located in an annulus fibrosis surrounding an intervertebral disc space using an inflatable annulus repair device having a central body having an external surface and a first diameter in an expanded state, an intradiscal region having a first disc wall surface and a second diameter in the expanded state, and an extradiscal region having a second disc wall surface and a third diameter in the expanded state, the annulus defect having side surfaces extending between interior and exterior annular walls, the method including the steps of:
a) forming an incision;
b) inserting a cannula into the incision so that a distal end of the cannula is positioned adjacent to the annulus defect;
c) inserting the inflatable annulus repair device in a first non-expanded state through the cannula and at least partially into the annulus defect so that the intradiscal region extends through the annulus defect and into the intervertebral disc space; and
d) injecting a filler material into the inflatable annulus repair device so that the external surface contacts the side surfaces, the first disc wall contacts the interior annular wall and the second disc wall contacts the exterior annular wall to seal the annulus defect and to apply a compression force to a captured portion of the annulus adjacent the defect in an implanted position.

21. The method of claim 20, further including the step of expanding the second diameter of the intradiscal region to a larger diameter than the first diameter of the central body.

22. The method of claim 20, further including the step of expanding the third diameter of the extradiscal region to a larger diameter than the first diameter of the central body.

23. The method of claim 20, further comprising the step of performing a discectomy on the intervertebral disc space prior to step (c).

24. The method of claim 20, further comprising the step of:
e) inflating one or more deployable secondary fixation members.

25. The method of claim 20, further comprising the step of:
e) permitting the filler material to solidify from a liquid at room temperature to an elastic at body temperature.

26. The method of claim 20, wherein the filler material is a thermogelling or phase transforming polymer.

27. The method of claim 20, further comprising the step of:
e) cutting off any excess length of the inflatable annulus repair device that protrudes beyond the defect so that the inflatable annulus repair device is generally flush with or recessed relative to the exterior annular wall.
